(19) United States
(12) Patent Application Publication
(10) Pub. No.: US 2015/0174360 A1
(43) Pub. Date: Jun. 25, 2015

(54) Device for Discharging Toxic Gases

(71) Applicant: 12th Man Technologies, Inc., Garden Grove, CA (US)

(72) Inventors: Alex Stenzler, Long Beach, CA (US); Steve Han, Huntington Beach, CA (US)

(21) Appl. No.: 14/581,059

(22) Filed: Dec. 23, 2014

Related U.S. Application Data
(60) Provisional application No. 61/920,203, filed on Dec. 23, 2013.

(51) Int. Cl. A61M 16/12; A61M 16/08; A61M 16/00

(52) U.S. Cl. CPC A61M 16/127; A61M 16/009; A61M 16/0816; A61M 2202/0233; A61M 2202/0275; A61M 2250/00

(57) ABSTRACT

A device for reducing the concentration of a gas from a gas source is described. The device includes a housing having a proximal end, a distal end and a length therebetween that at least partially encloses an interior region, wherein the proximal end has a gas inlet connectable to a gas source, and the distal end has a diluted gas outlet, a nozzle having at least one gas outlet, wherein the nozzle extends within the interior region of the housing, and wherein the nozzle gas outlet is fluidly connected to the housing gas inlet such that gas entering the housing gas inlet exits through the nozzle gas outlet, and at least one opening in the housing length, wherein the opening is adjacent to the at least one nozzle gas outlet and permits air to enter the housing interior region near the nozzle gas outlet. The device is incorporated into a gas delivery assembly, whereby excess gas can be safely purged from the assembly at a diluted concentration via the device.

### CROSS-REFERENCE TO RELATED APPLICATIONS

This application is entitled to priority under 35 U.S.C. §119(e) to U.S. provisional application No. 61/920,203, filed Dec. 23, 2013, which is incorporated herein by reference in its entirety.

### BACKGROUND OF THE INVENTION

Certain toxic gases, such as nitric oxide, carbon monoxide and hydrogen sulfide, are known to have therapeutic benefits at low concentrations, and can be used in applications for treating humans and animals. However, while administration of these toxic gases at low concentrations is safe for patient or healthcare worker exposure, unintended exposure to higher concentrations of these gases may be harmful.

Such gases are typically housed in a container, such as a cylinder, with a single port for filling and releasing the gas from the cylinder. In some situations, the container may need to be disconnected from a gas delivery device while there is still some gas remaining in the container. In addition, in some situations, the delivery line of the delivery device may need to be purged when gas has been left standing in the line. For example, nitric oxide can convert to nitrogen dioxide, a highly toxic gas in low concentrations, when it has been left in the delivery line between uses. Preventing unwanted exposure to higher concentrations of a gas when the user is disconnecting a cylinder with a standard valve is fairly easy. However, when the cylinder is of a disposable design, for example with a puncture seal and no valve, the risk of exposure is significantly greater.

Further, regardless of the design of the container, there is presently no particularly safe way to purge the delivery line, nor is there a way to discharge a cylinder containing residual gas, so that the gas concentration at the discharge point is low enough so as to not be harmful. For example, instructions for the emptying of gas cylinders or cartridges, or the purging of delivery lines, typically only state that the flow of gas should be directed away from people working with it. There are no “built-in” mechanisms or devices to improve the safety of gas discharge.

Thus, there is a need in the art for a device or method for safely emptying gas cylinders or cartridges of potentially toxic gases, particularly in cases where the gas cylinders or cartridges do not have valves. In addition, there is a need for a safe way to purge the delivery lines of a gas delivery device. The present invention satisfies this need.

### SUMMARY OF THE INVENTION

The present invention relates to devices and systems for reducing the concentration of a gas from a gas source. The present invention also relates to methods for reducing the concentration of a gas from a gas source or for purging a pressurized gas from a gas delivery device or system. In one embodiment, the gas being diluted or reduced in concentration is a toxic gas.

In one embodiment, the device of the present invention comprises: a housing having a proximal end, a distal end and a length therebetween that at least partially encloses an interior region, wherein the proximal end has a gas inlet connectable to a gas source, and the distal end has a diluted gas outlet; a nozzle having at least one gas outlet, wherein the nozzle extends within the interior region of the housing, and wherein the nozzle gas outlet is fluidly connected to the housing gas inlet such that gas entering the housing gas inlet exits through the nozzle gas outlet; and at least one opening in the housing length, wherein the opening is adjacent to the at least one nozzle gas outlet and permits air to enter the housing interior region near the nozzle gas outlet.

In one embodiment, the system of the present invention comprises: a gas source; a gas delivery device connected to the gas source, such that gas is capable of flowing from the gas source to the gas delivery device; a venturi device connected to the gas delivery device, such that gas is capable of flowing from the gas delivery device to the venturi device, and wherein the venturi device dilutes the concentration of gas flowing therethrough; wherein the gas delivery device is purged of excess gas when the flow of gas is directed to the venturi device.

In another embodiment, the system of the present invention is a system for purging gas from a gas source feeding a gas delivery device assembly, comprising: a gas source; a gas delivery device connected to the gas source, such that gas is capable of flowing from the gas source to the gas delivery device; a venturi device connected to the gas source, such that gas is capable of flowing from the gas source to the venturi device, and wherein the venturi device dilutes the concentration of gas flowing therethrough; and a valve, wherein the valve is capable of directing the flow of gas from the gas source to either the gas delivery device or the venturi device; wherein the assembly is purged of excess gas when the valve directs the flow of gas to the venturi device.

In one embodiment, the venturi device of the system of the present invention comprises: a housing with a proximal end, a distal end and a length therebetween that at least partially encloses an interior region, wherein the proximal end has a gas inlet connectable to the flow of gas from the gas delivery device, and the distal end has a diluted gas outlet; a nozzle having at least one gas outlet, wherein the nozzle extends within the interior region of the housing, and wherein the nozzle gas outlet is fluidly connected to the housing gas inlet such that gas entering the housing gas inlet exits through the nozzle gas outlet; and at least one opening in the housing length, wherein the opening is adjacent to the at least one nozzle gas outlet and permits air to enter the housing interior region near the nozzle gas outlet; wherein, when gas flows through the venturi device housing gas inlet and out the at least one nozzle gas outlet, room air is pulled into the device housing interior region and mixes with the gas, such that a diluted gas exits the device through the distal end outlet. In one embodiment, the venturi device of the system is detachable.

In one embodiment, the method of the present invention is a method for purging a pressurized gas from a gas delivery device comprises directing a flow of pressurized gas into the housing gas inlet of the venturi device of the present invention, wherein the gas exiting the device is diluted.

In one embodiment, the device, system, or method of the present invention can dilute the gas from a gas source to about 5% of its original concentration. In another embodiment, the device, system, or method of the present invention can dilute the gas from a gas source to less than 5% of its original concentration. In yet another embodiment, a toxic gas is diluted to a non-toxic concentration. In one embodiment, the gas source of the device, system, or method of the present invention is a gas cylinder.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A and 1B, is a schematic diagram of an exemplary embodiment of the venturi device of the present invention.

FIG. 2 is a schematic diagram showing the flow pattern of gas and air through an exemplary embodiment of the venturi device of the present invention.

FIG. 3 is a schematic diagram of a gas delivery system, including the venturi device of the present invention.

FIG. 4 is another schematic diagram of a gas delivery system, including the venturi device of the present invention.

FIG. 5 is yet another schematic diagram of a gas delivery system, featuring an embodiment of a detachable venturi device of the present invention.

FIG. 6 is a photo image of the venturi device of the present invention.

FIG. 7 is a schematic diagram showing the flow pattern of gas and air through an exemplary embodiment of the venturi device of the present invention.

FIG. 8 is a schematic diagram showing an experimental setup for evaluating an exemplary embodiment of the venturi device of the present invention.

FIG. 9 is a graph depicting sampling data from an experimental evaluation of an exemplary embodiment of the venturi device of the present invention.

FIG. 10 is a graph depicting sampling data from an experimental evaluation of an exemplary embodiment of the venturi device of the present invention.

FIG. 11 is a graph depicting sampling data from an experimental evaluation of an exemplary embodiment of the venturi device of the present invention.

FIG. 12 is a graph depicting sampling data from an experimental evaluation of an exemplary embodiment of the venturi device of the present invention.

FIG. 13 is a graph depicting sampling data from an experimental evaluation of an exemplary embodiment of the venturi device of the present invention.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical gas discharge systems. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The device and system of the present invention includes a venturi discharge port in line with a gas cylinder that utilizes the high pressure of the gas in the cylinder to generate a venturi effect that entrains room air and dilutes the gas from the cylinder, so that the discharged concentration of gas is diluted to safe concentrations before entering the surrounding environment. For example, in conjunction with a gas delivery device used for a medical application, the present invention may suitably dilute the undelivered, discharged gas escaping the gas delivery device, such that the discharged gas is within National Institute for Occupational Safety and Health (NIOSH) standards for respiratory exposure to those in close proximity.

In one embodiment, the venturi device of the present invention can be integrated into a gas delivery device or system. In such an embodiment, the venturi device can be connected to the system such that gas can be directed to the venturi device via a valve. In another embodiment, the venturi device can be a detachable device that is readily attached to and removed from a port or adaptor on a gas delivery device or system.

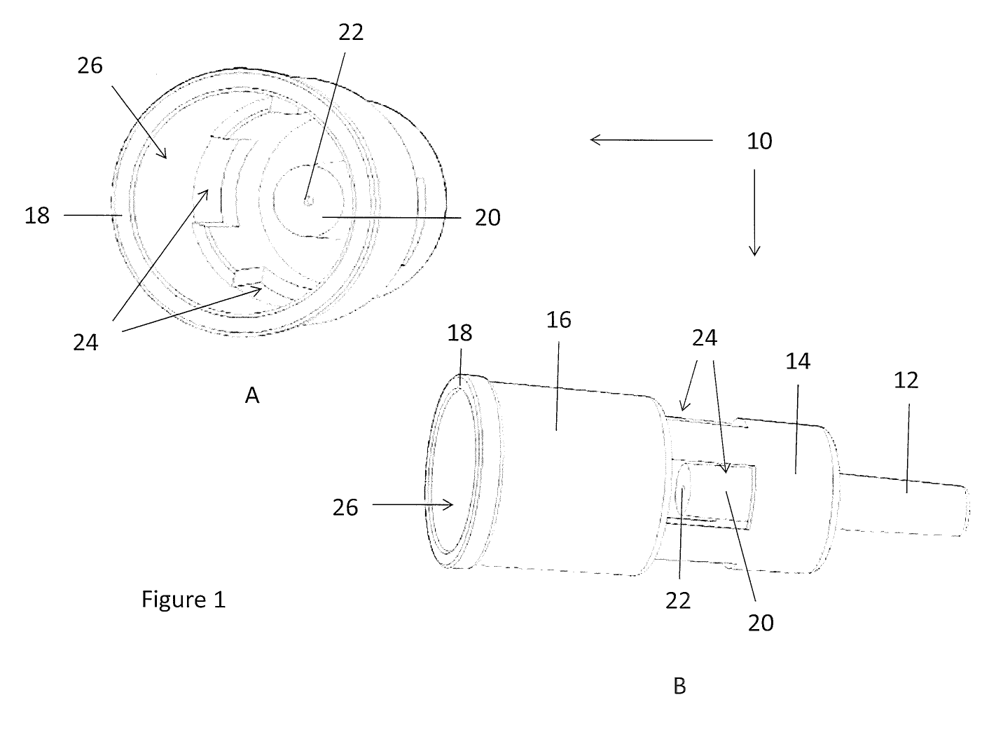

Referring now to FIGS. 1A and 1B, an exemplary venture device 10 of the present invention is shown. Venturi device 10 includes a proximal housing portion 12 for engagement with a gas source; a central housing portion 14 which includes windows or openings 24 for inflow of room air; a distal housing portion 16 that forms a cup to promote mixing of air and gas from the gas source; a distal end ring 18 of housing cup 16; a nozzle 20 with a gas outlet 22; and a passage 26 through distal housing portion 16 where air and gas from the gas source can mix.

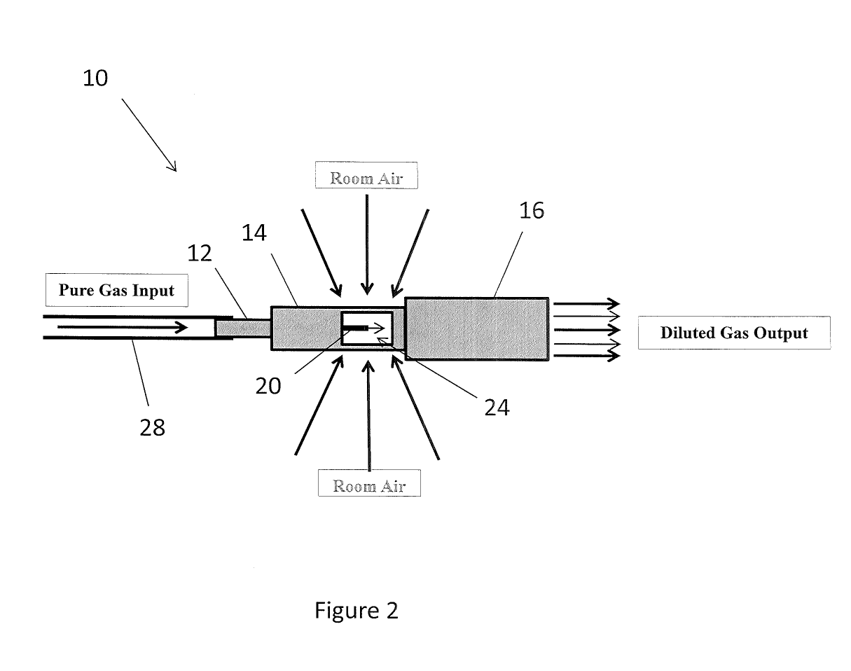

Referring now to FIG. 2, a schematic diagram depicting the flow of gas and/or air through venture device 10 of the present invention is shown. Gas from a gas source enters proximal housing portion 12 of venturi device 10 through tube/conduit 28. The gas then continues to central housing portion 14 where it exits nozzle 20 through the nozzle gas outlet 22. Upon exiting nozzle 20, the gas is mixed with air via a venturi effect, wherein air is pulled into venturi device 10 from the surrounding environment via entrainment windows/openings 24. The gas is thereby diluted with air to form a gas/air mixture that flows into and through distal housing portion 16. Distal housing portion 16 further serves to promote the mixing of the gas/air mixture such that the gas/air mixture exiting venturi device 10 is substantially uniform in concentration.

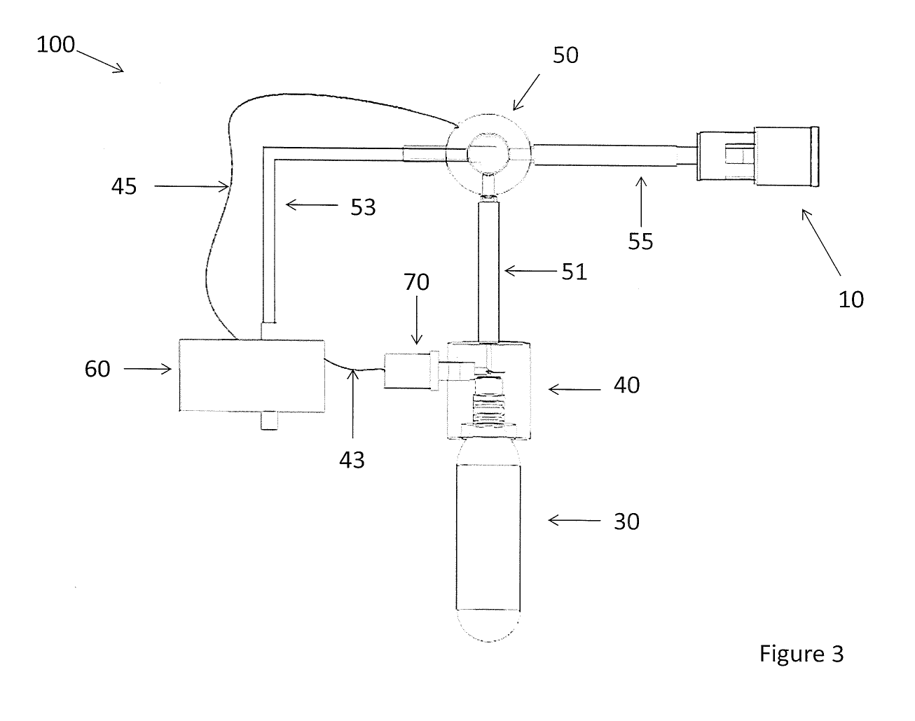

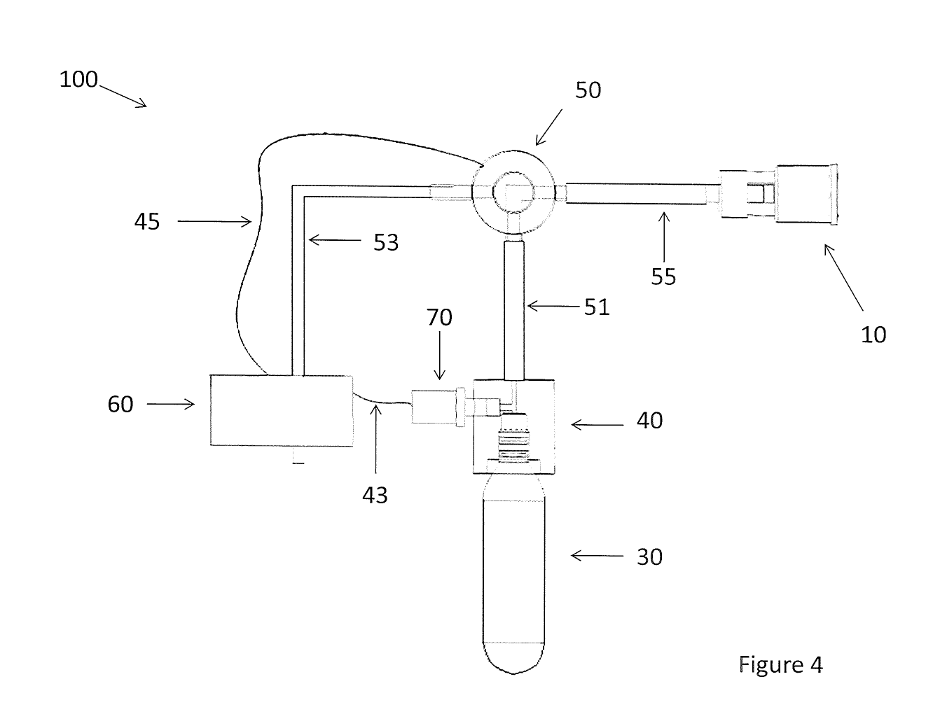

Referring now to FIG. 3, venturi device 10 of the present invention is shown connected to a gas delivery device 60 as part of a gas delivery system 100. A gas cylinder 30 is connected to a conduit 51 via a cylinder locking mechanism 40. Conduit 51 is connected to a conduit 53 via a three-way valve 50, wherein conduit 53 is further connected to gas delivery device 60. Three-way valve 50 is also connected to a conduit 55 to which venturi device 10 of the present invention is connected. In FIG. 3, three-way valve 50 is set to allow gas to flow from cylinder 30 into conduit 51, through valve 50, into conduit 53, and then into gas delivery device 60. System 100 can further include a pressure transducer 70 for sensing the pressure of gas in cylinder 30, or in other locations of the system. In addition, system 100 can include a controller. In one embodiment, such a controller can be integrated with gas delivery device 60 and connected to pressure transducer 70 and/or three-valve 50 via wires 43 and 45. In another embodiment, a controller can be connected wirelessly to pressure transducer 70 and/or three-valve 50. In one embodiment, three-way valve 50 can be automated. In such an embodiment, signals from controller/delivery device 60 can be sent to valve 50 to change the orientation of valve 50 to either the orientation shown in FIG. 3, or the orientation shown in FIG. 4, depending on whether gas needs to be sent from cylinder 30 to delivery device 60, or cylinder 30 needs to be emptied of any residual gas. In another embodiment, three-way valve 50 can be a manual valve, such that the orientation of the valve can be set manually by an operator.

Alternatively, as shown in FIG. 4, three-way valve 50 can be set to allow gas to flow from cylinder 30 into conduit 51, through valve 50, into conduit 55, and then through venturi device 10. Accordingly, when three-way valve 50 is set as shown in FIG. 4, any residual gas in cylinder 30 or in conduit line 51 can be discharged through venturi device 10 via conduits 51 and 55 safely into the surrounding environment. As previously described, any gas discharged from cylinder 30 and/or conduit 51 will be mixed with air via venturi device 10 to dilute the gas to a safe concentration prior to discharge into the environment.

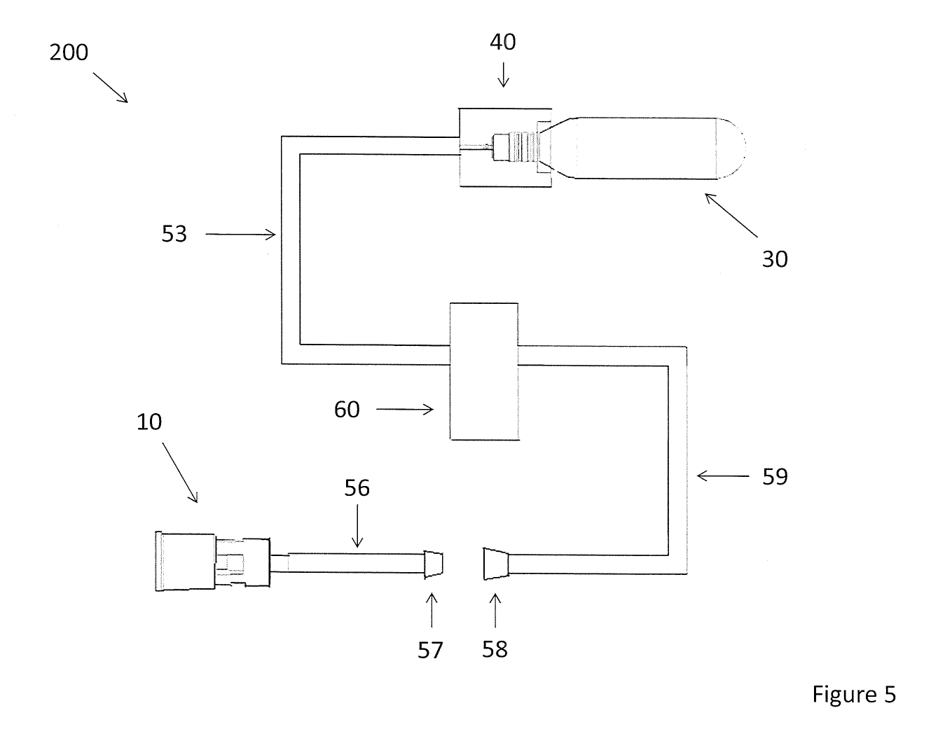

Referring now to FIG. 5, an embodiment of venturi device 10 is shown configured to another gas delivery system 200, wherein gas delivery system 200 does not include a three-way valve separate from the gas delivery device. Gas delivery system 200 includes a gas cylinder 30 connected to a gas delivery device 60 via cylinder locking mechanism 40 and conduit 53. Further, gas delivery system 200 includes a conduit 59 connected to gas delivery device 60. Conduit 59 has an adaptor 58 to which a component part can be attached, such as a mask. When gas delivery to a patient is complete, venturi device 10, having an adaptor 57 and conduit 56 can be connected to gas delivery system 200. In such an embodiment, venturi device 10 can be used to safely purge conduits 53 and 59 of system 200 by allowing gas to be diluted with air before being released to the surrounding environment. In one embodiment, adaptor 58 can include a valve such that the contents of conduits 59 and 53 are not vented to the environment until venturi device 10 is connected to system 200. Alternatively, venturi device 10 may be directly integrated into gas delivery device 60, such that an operator of gas delivery device 60 can simply initiate a “purge” function, whereby residual or excess gas is purged from gas delivery device 60 via the integrated venturi device 10.

In various embodiments, the venturi device can be sized so that a gas from the gas source, i.e., source gas, entering the venturi device is diluted with air form the surrounding environment to yield a source gas/air mixture having a safe concentration level of the source gas. In one embodiment, the venturi device is sized such that the source gas/air mixture exiting the venturi device comprises 5 percent or less source gas. However, the venturi device can be sized or modified such that the source gas/air mixture exiting the venturi device can comprise any concentration of source gas, as would be understood by a person skilled in the art. In various embodiments, the different components of the venturi device can be modified, i.e., made in a different size and/or shape, to change the gas mixing properties of the device. For example, in one embodiment, the size and/or shape of the nozzle 20 and nozzle outlet 22, and/or entrainment windows/openings 24 can be modified to yield a desired gas mixing property. In another embodiment, the number of entrainment windows/openings 24 can be increased or decreased to yield a desired gas mixing property. In yet another embodiment, the dimensions or shape of housing cup 16 can be modified to yield a desired gas mixing property.

In various embodiments, the venturi device can perform the mixing of a source gas with air regardless of the pressure of the source gas entering the venturi device. In one embodiment, the venturi device can adequately mix the source gas with air to yield a safe concentration of source gas in the source gas/air mixture for any pressure of source gas greater than atmospheric pressure, i.e., the pressure of air in the surrounding environment.

The venturi device of the present invention may be preferably constructed from standard polymer materials suitable for molding, as would be understood by those skilled in the art. Alternatively, the venturi device may be composed of a metal or metal alloy. It should be appreciated that the venturi device can be constructed as a single molded part, or it may be constructed as multiple parts for assembly.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

The following experiment was performed to measure the dilution capabilities of an exemplary embodiment of the venturi discharge system described herein, operating unrestricted at 30 PSI to determine if it can be used to safely discharge potentially toxic gases during purge or tank emptying functions in gas delivery systems.

100% carbon dioxide (CO₂) was used to simulate discharge of a toxic gas because of the availability of high speed CO₂ sensors. To test the dilution concentration of (CO₂) when diluting the gas using the present invention, the output concentrations were measured at different distances and positions from the gas output.

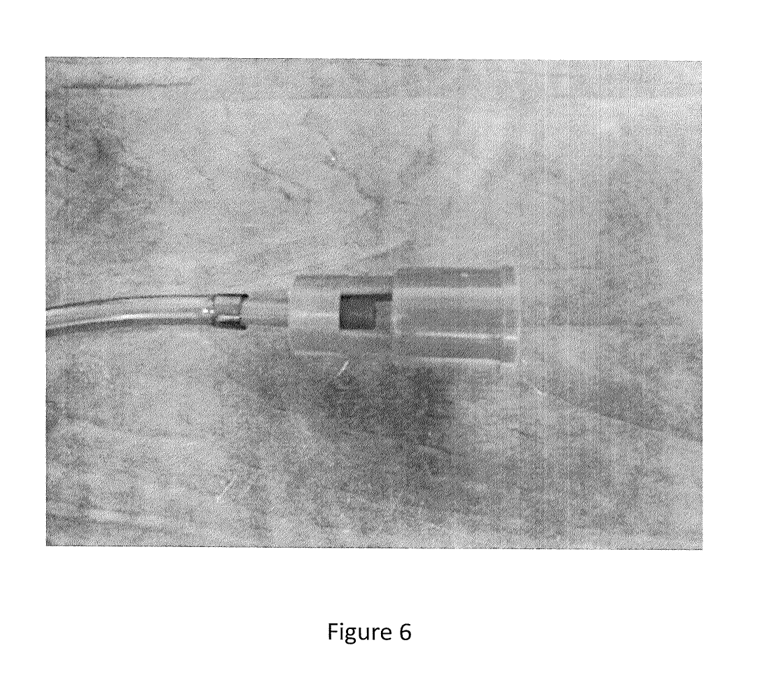

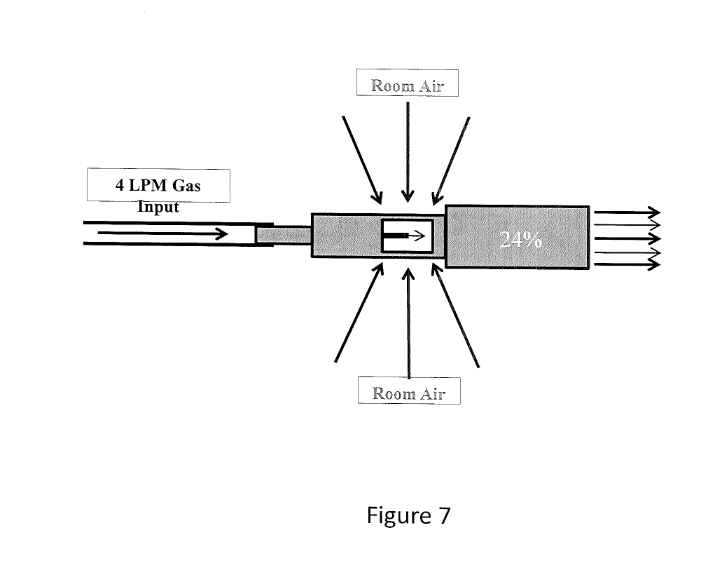

FIG. 6 is a photographic image of the present invention configured as an adapter attached to the gas source via a tube, as used for the experiment. A schematic of the adapter and its functionality is shown in FIG. 7. The inlet gas is converted into a high pressure, high-velocity jet at the convergent end of the nozzle, which creates a low pressure region at the nozzle surface. The low pressure draws room air into the adapter from the adapter flow inlet holes, where it mixes with the inlet gas. The design of this exemplary adapter at the rated flow of 4 liter per minute is designed for use with 100% oxygen and to entrain room air so that the resulting high flow output would be 24% oxygen. The nozzle flow would be 100% oxygen and the mixing with the 21% room air would result in 24% oxygen in the mixture. This adapter was designed to operate at a driving pressure of 50 PSI, while in the system where it would be used is pressurized at 30 PSI.

Using the calculations below, the 24% venturi adapter, described above, at 4 liters per minute of inlet flow entrains around 101.3 liters per minute of room air to get 24% of oxygen. This results in a total flow of 101.3+4 LPM input flow or 105.3 LPM. In the present example, these flow values were tested for a CO₂ venturi discharge system. It was contemplated that the flow values measured in the experimental CO₂ venturi discharge system would be substantially similar to the theoretical values calculated for an oxygen dilution system.

x LPM 100% O₂(cylinder)+y LPM 21% O₂(room atr)=z LPM 24% O₂

x×100|y×21=(4+y)×24

x=4 (inlet LPM)

4×100+y×21=(4+y)×24

400+21y=96+24y

304=3y

y=101.333

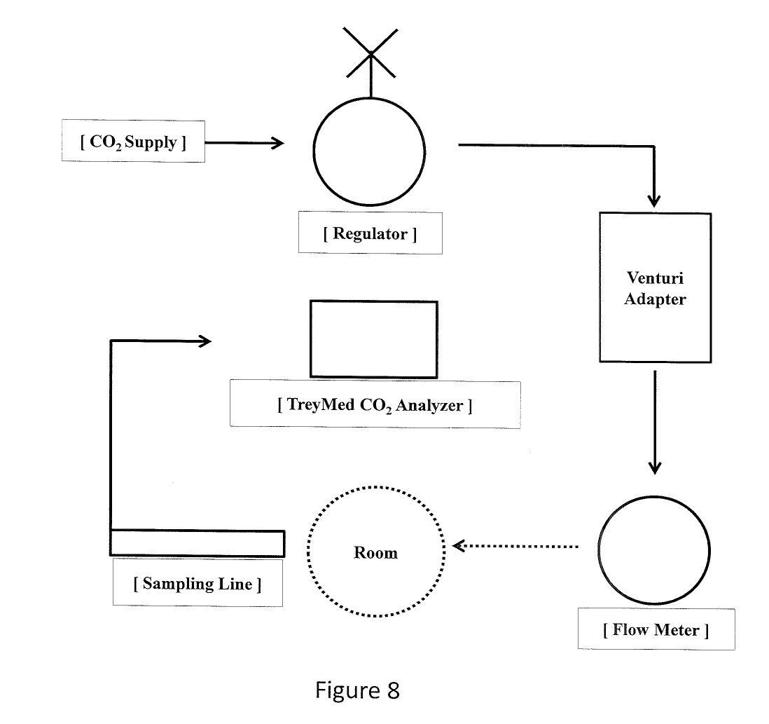

The experiment was set up as shown in FIG. 8. Tape was placed over the venturi adapter flow inlet holes to eliminate entrainment. A flow meter attached to the venturi adapter was turned on, and a 100% CO₂ supply was opened to the regulator, with the pressure regulator set at 30 PSI. Flow values from the flow meter were recorded when stabilized. The CO₂ supply was then fully closed, leaving the regulator unchanged. The flow meter was removed and the sampling line was connected to the CO₂ Analyzer. The CO₂ analyzer was turned on, and the sampling line inlet was placed 1 inch in front of the venturi adapter and perpendicular to the gas stream to sid-stream sample the gas. Data was then recorded with the CO₂ Analyzer components. The CO₂ supply was opened to the 30 PSI gas inlet pressure. After the CO₂ was stabilized, the CO₂ supply was closed. The process was repeated with the sampling line facing the gas stream at horizontal distances of 1, 2 and 3 feet away and again at the 1 foot distance and 1 foot vertically at the same position.

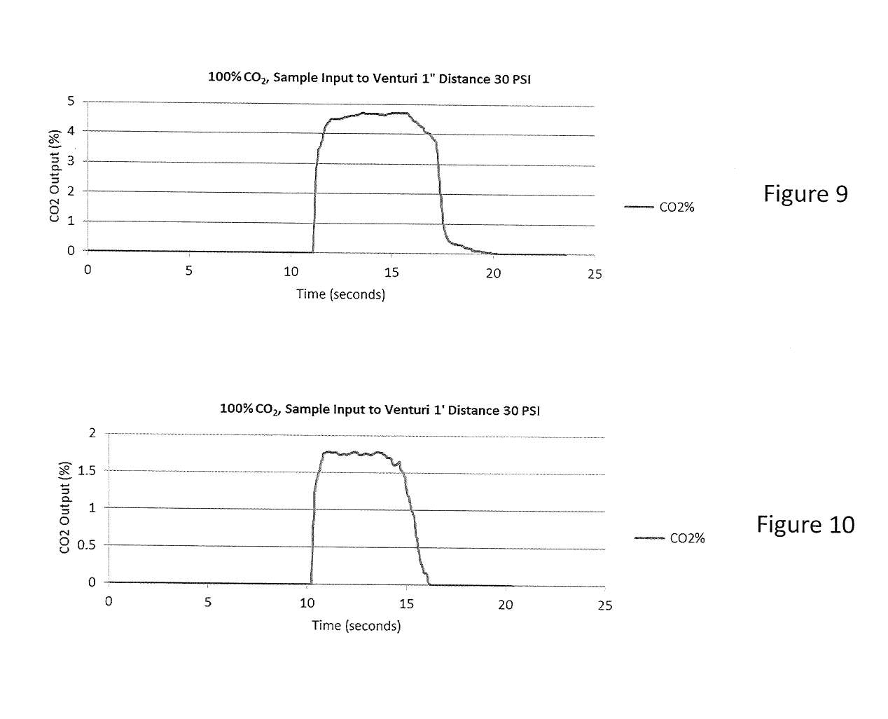

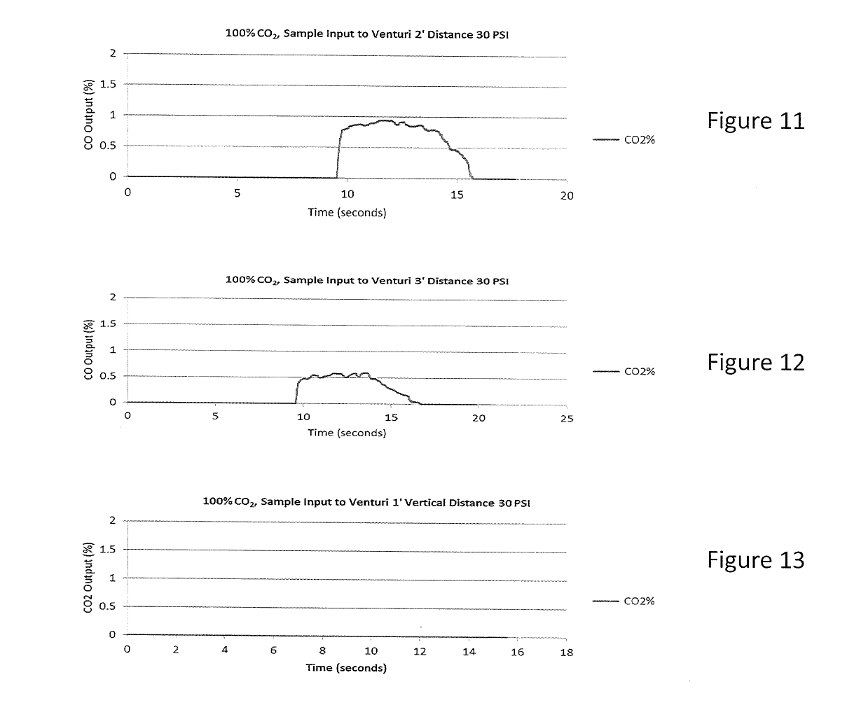

The results of this experiment, as shown in FIGS. 9-13 and Tables 1 and 2, demonstrate that the 24% Venturi adapter reduced the CO₂ output concentration to 4.72% when side-stream sampling the output gas at 1 inch in front of the venturi adapter output. When sampling at further distances, the CO₂ concentration decreased even more. Samples at one foot horizontal plus one foot vertical sampling distance, the measured CO₂ concentration level was near zero.

TABLE 1

| Set regulator pressure | Measured CO₂ flow |
|---|---|
| (PSI) | (liters per minute) |
| 30 | 13.4 |

TABLE 2

| | | Equivalent ppm concentration |
|---|---|---|
| | CO₂ Peak (%) | from 5000 ppm gas source |
| 1″ Horizontal Distance | 4.72 | 0.0236% = 236 ppm |
| 1′ Horizontal Distance | 1.78 | 0.0099% = 99 ppm |
| 2′ Horizontal Distance | 0.94 | 0.0047% = 47 ppm |
| 3′ Horizontal Distance | 0.58 | 0.0029% = 29 ppm |
| 1′ Vertical at 1′ Distance | 0.035 | 0.000175% = 1.75 ppm |

Based on the calculations presented above and the specifications for the venturi adapter, it was expected that the 100% CO₂ inlet gas would be diluted to the same extent as the 100% oxygen and would result in a diluted concentration of 3.797% (4 LPM/105.3 LPM) with the assumption that the 0.035% of CO₂ in room air can be considered negligible. However, the results obtained and reported in Table 2 (dilution to 4.72%) do not match this concentration value exactly. There are a couple of factors that could explain this difference. The CO₂ output peak measured at 1″ from the venturi adapter is higher than the expected value by +/−0.92%. The measured inlet flow to the venturi adapter was 13.4 LPM (Table 1) which is more than three times higher. Using higher flow rates can cause downstream turbulence, which could lead to less entrainment. Since less entrainment means relatively higher inlet gas flow, the measured gas concentration will also be higher. Other factors that could be considered to account for the measured higher concentration are: 30 PSI being used for the 50 PSI rated Venturi adapter; downstream configuration might reduce entrainment which can result in a higher outlet concentration, and; the venturi adapter is a molded part and manufacturing variations can affect the nozzle precision.

While the prediction of the resulting dilution was not exact, the test was conducted to determine the effectiveness of gas dilution using a venturi adapter of the present invention. In this regard, it was determined that with sampling using 100% CO₂ gas at a 1 inch distance of the output of the Venturi adapter, the output concentration was 4.72%. In comparison, when using a 5000 ppm (toxic) gas source, this would be diluted to +/−236 ppm (Table 2). At one foot it would be less than 100 ppm.

Accordingly, when using this 24% venturi adapter, the concentration of the inlet gas is diluted to what would be considered a safe level for inhalation exposure by the US Occupational Health and Safety Administration (OSHA) and is therefore an effective and useful gas discharge device and method for the purging of a gas delivery system and/or emptying small tanks of potentially toxic gases.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A device for reducing the concentration of a gas from a gas source, comprising:
a housing having a proximal end, a distal end and a length therebetween that at least partially encloses an interior region, wherein the proximal end has a gas inlet connectable to a gas source, and the distal end has a diluted gas outlet;
a nozzle having at least one gas outlet, wherein the nozzle extends within the interior region of the housing, and wherein the nozzle gas outlet is fluidly connected to the housing gas inlet such that gas entering the housing gas inlet exits through the nozzle gas outlet; and
at least one opening in the housing length, wherein the opening is adjacent to the at least one nozzle gas outlet and permits air to enter the housing interior region near the nozzle gas outlet.

2. The device of claim 1, wherein the gas from the gas source is diluted to about 5% or less of its original concentration.

3. A method for purging a pressurized gas from a gas delivery device, comprising directing a flow of pressurized gas into the housing gas inlet of the device of claim 1, wherein the gas exiting the device is diluted.

4. The method of claim 3, wherein the purged gas exiting the device is diluted to no more than about 5% of its original concentration.

5. A system for purging gas from a gas delivery device, comprising:
a gas source;
a gas delivery device connected to the gas source, such that gas is capable of flowing from the gas source to the gas delivery device;
a venturi device connected to the gas delivery device, such that gas is capable of flowing from the gas delivery device to the venturi device, and wherein the venturi device dilutes the concentration of gas flowing therethrough;
wherein the gas delivery device is purged of excess gas when the flow of gas is directed to the venturi device.

6. The system of claim 5, wherein the purged gas exiting the venturi device is diluted to no more than about 5% of its original concentration.

7. The system of claim 5, wherein the gas source is pressurized.

8. The system of claim 5, wherein the gas is a toxic gas.

9. The system of claim 8, wherein the gas exiting the venturi device is diluted to a non-toxic concentration.

10. The system of claim 5, wherein the venturi device comprises:
a housing with a proximal end, a distal end and a length therebetween that at least partially encloses an interior region, wherein the proximal end has a gas inlet connectable to the flow of gas from the gas delivery device, and the distal end has a diluted gas outlet;
a nozzle having at least one gas outlet, wherein the nozzle extends within the interior region of the housing, and wherein the nozzle gas outlet is fluidly connected to the housing gas inlet such that gas entering the housing gas inlet exits through the nozzle gas outlet; and
at least one opening in the housing length, wherein the opening is adjacent to the at least one nozzle gas outlet and permits air to enter the housing interior region near the nozzle gas outlet;
wherein, when gas flows through the venturi device housing gas inlet and out the at least one nozzle gas outlet, room air is pulled into the device housing interior region and mixes with the gas, such that a diluted gas exits the device through the distal end outlet.

11. A system for purging gas from a gas source feeding a gas delivery device assembly, comprising:
a gas source;
a gas delivery device connected to the gas source, such that gas is capable of flowing from the gas source to the gas delivery device;
a venturi device connected to the gas source, such that gas is capable of flowing from the gas source to the venturi device, and wherein the venturi device dilutes the concentration of gas flowing therethrough; and
a valve, wherein the valve is capable of directing the flow of gas from the gas source to either the gas delivery device or the venturi device;
wherein the assembly is purged of excess gas when the valve directs the flow of gas to the venturi device.

12. The system of claim 10, wherein the venturi device comprises:
a housing with a proximal end, a distal end and a length therebetween that at least partially encloses an interior region, wherein the proximal end has a gas inlet connectable to the gas source, and the distal end has a diluted gas outlet;
a nozzle having at least one gas outlet, wherein the nozzle extends within the interior region of the housing, and wherein the nozzle gas outlet is fluidly connected to the housing gas inlet such that gas entering the housing gas inlet exits through the nozzle gas outlet; and
at least one opening in the housing length, wherein the opening is adjacent to the at least one nozzle gas outlet and permits air to enter the housing interior region near the nozzle gas outlet;
wherein, when a pressurized gas flows through the device housing gas inlet and out the at least one nozzle gas outlet, room air is pulled into the device housing interior region and mixes with the gas, such that a diluted gas exits the device through the distal end outlet.

13. The system of claim 12, wherein the venturi device is detachable.

14. The system of claim 12, wherein the purged gas exiting the venturi device is diluted to no more than about 5% of its original concentration.

15. The system of claim 12, wherein the gas source is a gas cylinder.

16. The system of claim 14, wherein the gas is a toxic gas.

17. The system of claim 16, wherein the gas exiting the venturi device is diluted to a non-toxic concentration.
